(19) United States
(12) Patent Application Publication
(10) Pub. No.: US 2026/0157986 A1
(43) Pub. Date: Jun. 11, 2026

(54) METHODS OF TREATMENT OF PRIMARY BILIARY CHOLANGITIS

(71) Applicant: Ipsen Pharma, Boulogne-Billancourt (FR)

(72) Inventors: Benjamin MILLER, Cambridge, MA (US); Claudia ZEIN, Cambridge, MA (US); Jianfen SHU, Cambridge, MA (US)

(21) Appl. No.: 19/179,049

(22) Filed: Apr. 15, 2025

(30) Foreign Application Priority Data
Apr. 22, 2024 (EP) 24315205.5

(51) Int. Cl. A61K 31/192; A61K 31/575

(52) U.S. Cl. CPC A61K 31/192; A61K 31/575

(57) ABSTRACT

The present invention relates to elafibranor for use for the treatment of Primary Biliary Cholangitis (PBC) in a subject having PBC with a baseline plasma alkaline phosphatase (ALP) level of more than 1×ULN (Upper Limit Normal) and strictly less than t 1.67×ULN, before receiving elafibranor.

### FIELD OF THE INVENTION

The present invention relates to the field of medicine, in particular the treatment of Primary Biliary Cholangitis.

### BACKGROUND OF THE INVENTION

The present invention is dedicated to PBC (Primary Biliary Cholangitis, previously named Primary Biliary Cirrhosis), which is a cholestatic disease.

Cells in the liver produce bile, which passes through ducts within the liver to the gallbladder. Bile is a digestive liquid that is made in the liver. It travels through the bile ducts to the gallbladder and the small intestine, where it helps digest fats and fatty vitamins.

PBC is a chronic inflammatory intrahepatic, or long-term, liver disorder that slowly destroys the small-to-medium-sized bile ducts (tube-like structures that carry bile) within the liver. In patients with PBC, the bile ducts are destroyed by inflammation. This causes bile to remain in the liver, where gradual injury damages liver cells and causes cirrhosis, or scarring of the liver.

PBC is a rare, chronic, progressive autoimmune cholestatic liver disease characterized by lymphocytic cholangitis and associated with increased mortality. In untreated patients or those with an inadequate response to ursodeoxycholic acid (UDCA), PBC commonly progresses to fibrosis, cirrhosis, liver failure and death unless a liver transplant is received.

The estimated prevalence of PBC in North America, Europe, and the Asia Pacific region varies from 1.91 to 40.2 per 100,000 persons and the incidence varies from 0.23 to 5.31 per 100,000 persons, with the incidence increasing over time in North America and Europe. PBC predominantly affects women, with a female to male ratio of approximately 9:1. PBC is typically diagnosed between 40 and 60 years of age, and global estimates suggest that 1 in 1,000 females aged >40 years are living with PBC.

Approximately 60% of patients with PBC are asymptomatic at the time of diagnosis, often having been referred to a hepatologist for abnormal liver enzyme tests (most commonly, elevated alkaline phosphatase (ALP) or gamma glutamyl transferase (GGT) noted at the time of a routine health assessment. The majority of patients become symptomatic within 10 years of diagnosis. Initial presentation in symptomatic patients may include fatigue, pruritus, weight loss, right upper abdominal quadrant pain, and sometimes jaundice.

According to practice guidelines, a diagnosis of PBC is based on the presence of at least two of three of the following diagnostic criteria: an elevated serum ALP level (>1.5 times the upper limit of normal [ULN]), histologic evidence of chronic nonsuppurative biliary ductal destruction (florid duct lesion), and presence of anti-mitochondrial antibodies (AMA) in blood at a titer of 1:40 or greater. Liver biopsy is generally not required for diagnosis but can be useful in cases without a clear diagnosis, such as patients with negative AMA, or cases where differential diagnosis for potential clinically similar conditions, such as autoimmune hepatitis and non-alcoholic steatohepatitis (NASH) is needed.

In early PBC, patients are usually asymptomatic despite underlying inflammatory injury of small bile ducts, and slight anomalies in serum liver biochemical tests; this phase may continue for decades. An intermediate phase of PBC follows, where biochemical and clinical symptoms of cholestasis develop, while underlying lesions progress to ductopenia and liver fibrosis; this phase can continue for up to 10 years or more. In advanced PBC, patients may develop progressive jaundice, portal hypertension, and liver failure, sometimes deteriorating over the span of 2 to 4 years and progressing to liver related death in the absence of liver transplant. Hepatocellular carcinoma also may develop in advanced stage PBC.

Nearly all patients with PBC will become symptomatic during the course of their disease. Pruritus and fatigue are the most frequent symptoms. Other common symptoms include sicca complex, abdominal pain, arthralgia, restless legs, sleeplessness, depression and cognitive dysfunction.

Pruritus affects up to 70% of patients with PBC and can contribute to substantial morbidity. Because pruritus follows the circadian rhythm and is often worse at night, patients with PBC may also suffer from diminished sleep quality, leading to increased fatigue and a negatively impacted QoL. Pruritus may occur during early stages of the disease and may persist even in the setting of biochemical response or ALP normalisation following treatment with UDCA. Fatigue affects up to 80% of patients with PBC and also negatively affects QoL. Fatigue often interferes with the patient's ability to perform activities of daily living and is associated with diminished mental and physical capacity. While pruritus may improve with liver transplantation, fatigue frequently persists even after liver transplantation.

In adult patients with persistent biochemical cholestasis (e.g. elevation of alkaline phosphatase (ALP) in blood tests), a diagnosis of PBC can be made based on elevated ALP and the presence of antimitochondrial antibody (AMA). Current guidelines recommend against liver biopsy for the diagnosis of PBC, unless PBC-specific antibodies are absent, or there is a reason to suspect overlap with autoimmune hepatitis or co-existence of other liver diseases.

The rate of disease progression varies among individual patients, but in general progression is faster in patients who are inadequate responders to existing therapies, or in those who do not receive medical therapy.

The definition of inadequate response has traditionally been based on published definitions which centre around reduction of ALP levels below specific cut-offs. This is supported by the established knowledge that elevated ALP levels, along with elevated bilirubin levels, have been shown to be highly predictive of adverse long-term clinical outcomes, including need for liver transplant and death.

Ursodeoxycholic acid (UDCA) was the first treatment for PBC approved by regulatory authorities. UDCA improves ALP and bilirubin in blood, delays histological progression of disease and increases liver transplant-free survival. UDCA is recommended as the first line therapy for patients with PBC. However, up to 40% of patients with PBC have an inadequate response to UDCA therapy. Additionally, up to 5% of patients with PBC are intolerant to UDCA.

In recent years, obeticholic acid, a semi-synthetic analogue of the primary bile acid chenodeoxycholic acid, was approved as a second line therapy by regulatory authorities under the accelerated approval regulations. However, only a portion of patients with PBC who have not responded optimally to UDCA have an adequate response to added-on obeticholic acid, and in addition, clinically significant pruritus has been reported in a significant portion of patients. Importantly, improvement in survival or disease-related symptoms has not yet been clearly established. Furthermore, after reports of patients with advanced cirrhosis developing liver failure after treatment with obeticholic acid, in 2021 regulatory authorities restricted its use in patients with PBC advanced cirrhosis.

Data from the Global PBC Study Group showed that any elevation of ALP above 1× upper limit of normal (ULN) was associated with an increased risk of poor outcomes in patients with PBC, and that this risk was similar for patients with ALP elevations >1×ULN up to <1.67×ULN compared to those with elevations ≥1.67×ULN. Furthermore, any ongoing elevation in ALP after treatment with UDCA has been shown to be associated with ongoing disease activity in PBC. The higher survival rate in patients with ALP levels ≤ULN compared to patients with ALP>ULN was particularly noticeable among patients <62 years of age with advanced fibrosis based on liver stiffness measurement (LSM)≥10 kPa. These observations support the additional survival benefits of obtaining normalisation of serum ALP.

Although a portion of patients with PBC have an adequate response to available therapies, the vast number of patients with PBC do not have optimal response, or are intolerant to available therapies, and thus remain at risk of disease progression and long-term adverse clinical outcomes. Elafibranor (GFT505, IPN60190) is an orally administered dual PPARα,δ agonist being developed by Ipsen for the treatment of PBC in combination with UDCA in adults with an inadequate response to UDCA or as monotherapy in adults unable to tolerate UDCA.

Recently, the double-blind period of ELATIVE, a phase III double-blind, randomised, placebo-controlled study to evaluate the efficacy and safety of elafibranor in participants with PBC with inadequate response or intolerance to UDCA (GFT505B-319-1 (NCT04526665)), was completed and demonstrated that treatment with elafibranor resulted in greater improvement in cholestasis compared to placebo. This was assessed based on the primary endpoint, which combined reductions in ALP and total bilirubin (TB). Moreover, elafibranor is safe and well-tolerated by the patients.

Considering the efficacy and tolerability issues with the current treatment options available, a clear unmet need for second line therapy still remains, particularly for subjects with PBC with an inadequate response or intolerance to UDCA. Notably, the patients with PBC and baseline plasma ALP>1×ULN but <1.67×ULN traditionally represent a non-studied subset of patients: they were excluded from ELATIVE, as well as other Phase 3 studies with obeticholic acid and seladelpar. There is therefore an overlooked unmet need among these patients with PBC.

### SUMMARY OF INVENTION

A clinical study has surprisingly shown that the treatment of subjects with elafibranor provides a relevant reduction of biochemical markers in the plasma, demonstrating that this compound is advantageous for the treatment of PBC.

It has been also surprisingly shown that such compound is advantageous in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

The present invention relates to elafibranor for use for the treatment of Primary Biliary Cholangitis (PBC) in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

Another object of the invention is elafibranor for use for normalizing plasma ALP level in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

Another object of the invention is elafibranor for use for reducing plasma ALP level in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

Another object of the invention is a method of treating PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor, comprising administering to the subject in need thereof an effective dose of elafibranor.

Another object of the invention is a method of treating PBC comprising administering to a subject having PBC with a baseline plasma alkaline phosphatase (ALP) level of more than 1×ULN (Upper Limit Normal) and at most 1.67×ULN, in particular strictly less than 1.67×ULN, before receiving elafibranor, an effective dose of elafibranor.

In a particular embodiment of the invention, the subject to be treated is identified by testing the ALP level of a potential subject and then selecting the subject having baseline plasma ALP level of more than 1×ULN (Upper Limit Normal) and at most 1.67×ULN, in particular strictly less than 1.67×ULN, before receiving elafibranor.

In a particular embodiment of the invention, elafibranor is administered at a dose varying from 0.01 mg to 1 g per administration, preferentially from 1 mg to 150 mg per administration, more preferably from 70 mg to 130 mg, and more preferably at a dose of 80 mg.

In a particular embodiment, elafibranor is administered once a day.

In another particular embodiment, elafibranor is administered once a day at a dose of 80 mg, preferably in combination with UDCA to a subject with PBC and inadequate response to UDCA. The present invention also provides the use of elafibranor in the preparation of a pharmaceutical composition, preferably an oral pharmaceutical composition, for treating PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

According to the invention, the pharmaceutical composition may be formulated in the form of injectable suspensions, gels, oils, pills, tablets, suppositories, powders, gel caps, capsules, aerosols or means of galenic forms or devices assuring a prolonged and/or slow release.

According to the present invention, the disclosed method, compound, uses, composition or kit concern the treatment of PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

### Abbreviations and Definitions

In the context of the present invention, the following abbreviations, definitions, and empirical formulae are used in the figures, in the tables, and in the text.

### Abbreviations

ALP Alkaline Phosphatase
ALT Alanine Transaminase
AMA Anti-mitochondrial antibody
ASBTi Apical sodium-codependent bile acid transporter inhibitors
AST Aspartate Aminotransferase
C4 Serum 7a-hydroxy-4-cholesten-3-one
CDCA Chenodeoxycholic acid
CPK Creatine phosphokinase
DCA Deoxycholic acid
ECG Electrocardiogram
eGFR Estimated glomerular filtration rate
FGF19 Fibroblast growth factor 19
FXR Farnesoid X receptor
GGT Gamma glutamyl transferase
HBV Hepatitis B virus
HDL-C High Density Lipoprotein-Cholesterol
HIV Human immunodeficiency virus
IgM Immunoglobulin M
IL-20 Interleukin-20
LDL-C Low Density Lipoprotein-Cholesterol
MELD Model for End Stage Liver Disease
MRI Magnetic resonance imaging
NASH Nonalcoholic steatohepatitis
NOX NADPH oxidase
OCA Obeticholic acid
PBC Primary Biliary Cholangitis
PK Pharmacokinetics
PPAR Peroxisome proliferator-activated receptor
PT-INR Prothrombin Time—International Normalized Ratio
QOL Quality of life
TB Total bilirubin
TG TriGlyceride
TGF Transforming growth factor
UDCA Ursodeoxycholic acid
ULN Upper limit of normal
VAS Visual analogue score

### Definitions

The term “an effective amount” or “therapeutic effective amount” refers to an amount of the compound sufficient to produce the desired therapeutic result and elafibranor is administered in amounts that are sufficient to display the desired effect.

In the context of the invention, the terms “baseline plasma level” refer to the plasma level of a subject before starting any treatment with the composition of the invention comprising elafibranor. For example, “baseline ALP plasma level” refer to the ALP plasma level of a subject before starting any treatment with the composition of the invention comprising elafibranor.

In the context of the invention, the terms “before receiving elafibranor” means before the first ever administration of elafibranor to a subject. In other words, the subject having PBC with a baseline plasma alkaline phosphatase (ALP) level of more than 1×ULN (Upper Limit Normal) and at most 1.67×ULN, in particular strictly less than 1.67×ULN, has never been treated with elafibranor.

The term “patient”, “subject” or “individual” are interchangeable and refer to a mammal and more particularly a human including adult and child. The subjects to be treated according to the invention can be appropriately selected on the basis of several criteria associated with PBC such as previous and/or present drug treatments, associated pathologies, genotype, exposure to risk factors, as well as any other relevant biomarker that can be evaluated by means of any suitable immunological, biochemical, or enzymatic method.

As used herein, the term “pharmaceutical composition” refers to an active compound, or pharmaceutically acceptable salt thereof, formulated together with one or more pharmaceutically acceptable carriers. In some embodiments, active compound or salt is present in unit dose amount appropriate for administration in a therapeutic regimen that shows a statistically significant probability of achieving a predetermined therapeutic effect when administered to a relevant population. In some embodiments, pharmaceutical compositions may be specially formulated for parenteral administration, for example, by subcutaneous, intramuscular, intravenous or epidural injection as, for example, a sterile solution or suspension, or slow-release formulation. In other embodiments, pharmaceutical compositions include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). In other embodiments, pharmaceutical compositions include those formulated for topical administration (e.g., as a cream, gel, lotion, or ointment). Other pharmaceutically acceptable excipients can be colorants, flavoring agents, plasticizers, humectants, buffering agents, and the like. Compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

The term “treatment” or “treating” refers to therapy, prevention, or prophylaxis of PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

Other features, properties and advantages of the invention will emerge more clearly from the description and examples that follow.

### DETAILED DESCRIPTION

As mentioned above, an object of the present invention is elafibranor for use for the treatment of PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

Another object of the present invention is elafibranor for use for reducing plasma ALP level in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

In a particular embodiment, elafibranor can be used for normalizing ALP, albumin and/or TB level(s) in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

In a particular embodiment, the subject has PBC and responds at least partly to UDCA.

In another embodiment, the subject has PBC and does not respond adequately to UDCA.

In the context of the invention, the terms “does not respond adequately to UDCA” or “have inadequate response to UDCA” means that the plasma ALP level of the subject with inadequate response to UDCA remains above the ULN despite being treated with UDCA.

In another embodiment, the subject has PBC and is intolerant to UDCA.

In a particular embodiment, elafibranor is administered, preferably orally, to a subject with PBC and inadequate response to UDCA, in particular at a dose of 80 mg or 120 mg.

In a particular aspect, the desired effect is an improvement in alkaline phosphatase and/or GGT levels signing a reduction in cholestasis. Accordingly, the invention also relates to elafibranor for use in the improvement of ALP and/or GGT levels in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

In a particular embodiment, elafibranor is administered to a subject having PBC with a baseline plasma alkaline phosphatase (ALP) level of more than 1×ULN (Upper Limit Normal) and at most 1.67×ULN, in particular strictly less than 1.67×ULN, before receiving elafibranor, for normalising plasma ALP, albumin and/or bilirubin level(s).

In a particular embodiment, the subject has PBC with a baseline plasma alkaline phosphatase (ALP) level of more than 1×ULN (Upper Limit Normal) and at most 1.67×ULN, in particular strictly less than 1.67×ULN, before receiving elafibranor, and the treatment results in a level of ALP equal or lower than 1×ULN, and preferably lower than 0.5×ULN.

In a particular variant of this embodiment, elafibranor is administered for decreasing ALP level by at least 15%.

In a particular variant of this embodiment, elafibranor is administered for decreasing ALP level by at least 40%.

In a particular embodiment, the subject has PBC with a baseline plasma alkaline phosphatase (ALP) level of more than 1×ULN (Upper Limit Normal) and at most 1.67×ULN, in particular strictly less than 1.67×ULN, before receiving elafibranor, and the treatment results in a level of total bilirubin (TB) lower than 0.7×ULN. The reference range of total bilirubin is 0.2-1.2 mg/dL. The reference range of direct bilirubin is 0.1-0.4 mg/dL.

In a particular embodiment, the subject has PBC with a baseline plasma alkaline phosphatase (ALP) level of more than 1×ULN (Upper Limit Normal) and at most 1.67×ULN, in particular strictly less than 1.67×ULN, before receiving elafibranor, and the treatment results in a level of ALP equal or lower than 1×ULN, preferably lower than 0.5×ULN, and total bilirubin (TB) lower than 0.7×ULN. In a particular embodiment, the subject has PBC with a baseline plasma alkaline phosphatase (ALP) level of more than 1×ULN (Upper Limit Normal) and at most 1.67×ULN, in particular strictly less than 1.67×ULN, before receiving elafibranor, and the treatment results in a normalisation of the plasma ALP level, in a level of ALP equal or lower than 0.5×ULN, in a reduction of ALP level by at least 40%, and total bilirubin (TB) lower than 0.7×ULN.

In a particular embodiment, elafibranor is administered to a subject having PBC with a baseline plasma alkaline phosphatase (ALP) level of more than 1×ULN (Upper Limit Normal) and at most 1.67×ULN, in particular strictly less than 1.67×ULN, before receiving elafibranor, to improve bile acids level such as CDCA, cholic acid, litocholic acid and deoxycholic acid DCA levels.

In a further embodiment, elafibranor is administered for improving Paris I, Paris II, Toronto I, Toronto II or UK-PBC risk score.

In another embodiment, elafibranor is administered to a subject having PBC with a baseline plasma alkaline phosphatase (ALP) level of more than 1×ULN (Upper Limit Normal) and at most 1.67×ULN, in particular strictly less than 1.67×ULN, before receiving elafibranor, for:
improving AST, GGT, 5′-nucleotidase, total bilirubin, conjugated bilirubin, ALT and albumin levels;
improving lipid parameters;
improving C4 and/or FGF19 levels;
improving IgM levels; and/or
improving 5D-itch scale, PGI-S scores, PGI-C scores, PBC 40 QOL, VAS.

The treatment involves the administration of elafibranor (such as via the administration of a pharmaceutical composition comprising elafibranor) to a subject (e.g. a patient) having PBC with a baseline plasma alkaline phosphatase (ALP) level of more than 1×ULN (Upper Limit Normal) and at most 1.67×ULN, in particular strictly less than 1.67×ULN, before receiving elafibranor to prevent, cure, delay, reverse, or slow down the progression of the disease, improving thereby the condition of patients.

A treatment may be also administered to subjects that are either healthy or at risk of developing a cholestatic disease.

The subject to be treated is with PBC, as characterized as follows:
the presence of at least 2 of the following 3 diagnostic factors:
(i) history of elevated ALP levels for at least 6 months prior to Day 0 (randomization visit)
(ii) positive Anti-Mitochondrial Antibodies (AMA) titers (>1/40 on immunofluorescence or M2 positive by enzyme-linked immunosorbent assay (ELISA) or positive PBC-specific antinuclear antibodies
(iii) liver biopsy consistent with PBC
1×ULN<ALP≤1.67×ULN, in particular 1×ULN<ALP<1.67×ULN
optionally, taking UDCA for at least 12 months (stable dose for ≥6 months) prior to screening visit.

The present invention further relates to elafibranor, for use in a method for reducing plasma ALP level in a subject having PBC with a baseline plasma ALP level of more than 1×ULN and at most 1.67×ULN, in particular strictly less than 1.67×ULN, before receiving elafibranor.

In the context of the invention, the terms “reducing plasma ALP level in a subject” means either a reduction of at least 15% of the plasma ALP level of said subject compared to the baseline plasma ALP level or a reduction of the plasma ALP level of the subject to no more than 1×ULN, after 52 weeks of treatment with elafibranor. Reducing plasma ALP level in a subject having PBC helps to improve the condition of said subject, in particular signing a reduction in cholestasis.

In the context of the invention, the terms “normalizing ALP level in a subject” means reaching sustained normal ALP levels (i.e. below ULN) after initial normalization. Normalizing plasma ALP level in a subject having PBC helps to improve the condition of said subject, in particular signing a reduction in cholestasis.

In the context of the invention, ALP results are reported in international units per liter (IU/L or U/L). For males older than age 18, the upper limit of normal (ULN) related to the ALP level is 129 U/L. For females older than age 18, the upper limit of normal (ULN) related to the ALP level is 104 U/L. In a particular embodiment of the invention, the subject to be treated is identified by testing the ALP level of a potential subject and then selecting the subject having baseline plasma ALP level of more than 1×ULN and at most 1.67×ULN, in particular strictly less than 1.67×ULN, before receiving elafibranor. The ALP level is testing using methods commonly known in the art. An ALP test requires a blood sample from a vein.

In the context of the invention, AST results are reported in international units per liter (IU/L or U/L). For males older than age 18, the upper limit of normal (ULN) related to the AST level is 40 U/L. For females older than age 18, the upper limit of normal (ULN) related to the AST level is 32 U/L. According to a particular embodiment, the subject has Child-Pugh score A.

According to a particular embodiment, the subject has not Child-Pugh score B or C.

Liver cirrhosis is defined as compensated or decompensated and further classified using the Child-Pugh system which is well known to individuals skilled in the art. Cirrhosis patients are classified on the basis of certain clinical parameters. Child Pugh A patients are compensated and may display minimal obvious symptoms. Patients classified as Child Pugh B or Child Pugh C are decompensated and can exhibit outward symptoms such as ascites.

The Child-Pugh score consists of five clinical features including, ascites, hepatic encephalopathy, albumin, total bilirubin and PT-INR and is used to assess the prognosis of chronic liver disease and cirrhosis. Each component is given a numerical score from 1 to 3 and added to provide total scores ranging from 5 to 15. The higher the score the worse prognosis is for the patient. Patients with a total score of 5-6 are classified as Child Pugh A. Patients with a total score of 7-9 are classified as Child Pugh B. Patients with a total score of 10-15 are the most ill and classified as Child Pugh C.

The Child-Pugh score was originally developed in 1973 to predict surgical outcomes in patients presenting with bleeding esophageal varices. Several studies have shown that Child-Pugh score is an independent prognostic marker in the settings of several diseases including Primary Biliary Cholangitis (PBC). Child-Pugh score can be easily calculated at the bedside.

The components of the scoring system and the point allocations are listed in the table below.

| Measure | 1 point | 2 points | 3 points |
|---|---|---|---|
| Total bilirubin (mg/dl) | <2 | 2-3 | >3 |
| Serum albumin (g/dl) | >3.5 | 2.8-3.5 | <2.8 |
| Prothrombin time, | <4.0 | 4.0-6.0 | >6.0 |
| prolongation (s) | | | |
| Ascites | None | Mild | Moderate to Severe |
| Hepatic | None | Grade I-II (or | Grade III-IV (or |
| encephalopathy | | suppressed with | refractory) |
| | | medication) | |

The predicted 1-year survival based on Child Pugh score is presented in the table below:

| | Class A | Class B | Class C |
|---|---|---|---|
| Total Points | 5-6 | 7-9 | 10-15 |
| 1-year survival | 100% | 80% | 45% |

Elafibranor can have different stable isomeric forms.

Synthesis of elafibranor can for example be carried out as described for compound 29 in WO 2004/005233 and US 20050176808.

Elafibranor can be formulated as pharmaceutically acceptable salt, particularly an acid or base salt compatible with pharmaceutical use. Salts of elafibranor implemented herein include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound.

In particular, “pharmaceutically acceptable salts” include inorganic as well as organic acids salts. Counter-ions may be selected from the following the non-exhaustive list: ammonia, L-arginine, benethamine, benzathine, tert-butylamine (erbumine), calcium hydroxide, choline hydroxide, deanol, diethanolamine (2,2′-iminobis(ethanol), diethylamine, epolamine (1-(2-hydroxyethyl)pyrrolidine), 2-(diethylamino)-ethanol, ethanolamine (2-aminoethanol), ethylenediamine, glycine, hydrabamine, 1H-imidazole, L-Lysine, magnesium hydroxide, meglumine (N-methyl-glucamine), 4-(2-hydroxyethyl)-morpholine, piperazine, potassium hydroxide, sodium hydroxide, triethanolamine (2,2′,2″-nitrilo-tris(ethanol)), tromethamine, zinc hydroxide, in particular tromethamine, potassium, sodium, benethamine, benzathine, L-arginine, ethanolamine, meglumine, glycine, erbumine, L-lysine, epolamine, choline, preferably tromethamine, potassium, sodium, benethamine, benzathine, L-arginine, more preferably tromethamine, potassium, sodium, L-arginine, more particularly tromethamine.

In particular embodiments, the invention implements an ammonia, L-arginine, benethamine, benzathine, tert-butylamine (erbumine), calcium, choline, deanol, diethanolamine (2,2′-iminobis(ethanol), diethylamine, epolamine (1-(2-hydroxyethyl)pyrrolidine), 2-(diethylamino)-ethanol, ethanolamine (2-aminoethanol), ethylenediamine, glycine, hydrabamine, 1H-imidazole, L-Lysine, magnesium, meglumine (N-methyl-glucamine), 4-(2-hydroxyethyl)-morpholine, piperazine, potassium, sodium, triethanolamine (2,2′,2″-nitrilo-tris(ethanol)), tromethamine or zinc salt of elafibranor. In a further particular embodiment, the salt of elafibranor is selected from a tromethamine, potassium, sodium, L-arginine, benethamine, benzathine, ethanolamine, meglumine, glycine, erbumine, L-lysine, choline, epolamine, magnesium or 2-amino-2-methyl-propan-1-ol salt of elafibranor.

The pharmaceutical compositions comprising elafibranor for the treatment of PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor, can comprise one or several excipients or vehicles, acceptable within a pharmaceutical context (e.g. saline solutions, physiological solutions, isotonic solutions, etc., compatible with pharmaceutical usage and well-known by one of ordinary skill in the art). These compositions can comprise one or several agents or vehicles chosen among dispersants, solubilisers, stabilisers, preservatives, etc.

Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc.

These compositions can be formulated in the form of injectable suspensions, gels, oils, pills, suppositories, powders, gel caps, capsules, aerosols, etc., eventually by means of galenic forms or devices assuring a prolonged and/or slow release. For this kind of formulation, agents such as cellulose, carbonates or starches can advantageously be used.

Elafibranor may be administered in an efficient amount by using a pharmaceutical composition as above defined.

Elafibranor can be administered in different ways and in different forms that allow administering said compounds in a therapeutically effective amount.

Thus, for example, it can be administered in a systematic way, per os, by parenteral route, by inhalation, or by injection, such as for example intravenously, by intra-muscular route, by subcutaneous route, by transdermal route, by intra-arterial route, etc.

Oral administration is the preferential route of administration for pharmaceutical compositions comprising elafibranor for the treatment of PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

Suitable formulations, and in particular tablet formulations, are described in WO 2024/184365.

The frequency and/or dose relative to the administration can be adapted by one of ordinary skill in the art, in function of the patient, the pathology, the form of administration, etc. Typically, elafibranor can be administered for the treatment of PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor at doses varying from 0.01 mg to 1 g per administration, preferentially from 1 mg to 150 mg per administration, and more preferably from 70 mg to 130 mg.

Administration can be performed daily or even several times per day, if necessary.

In a particular embodiment, elafibranor is administered once a day.

In another particular embodiment, elafibranor is administered once a day at a dose of 80 mg or 120 mg, preferably at a dose of 80 mg.

In a particular embodiment, elafibranor is administered throughout the patient's life.

In a particular embodiment, elafibranor is not administered as sole therapeutically active agent.

Thus, in a particular embodiment, the invention also relates to the use of elafibranor for the treatment of PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor, in combination with at least one other therapeutically active agent.

The other active agent may in particular be selected from other anti-cholestatic agents such as UDCA or OCA.

The invention thus also relates to the combination of elafibranor with UDCA or OCA, and preferably with UDCA.

The invention also relates to the combination of elafibranor with an anti-cholestatic agent.

Other anti-cholestatic agents include, without limitation:
apical sodium-codependent bile acid transporter inhibitors (ASBTi);
bile acids;
cathepsin inhibitors;
CCR antagonists;
CD40 inhibitors;
CD80 inhibitors;
Dual NOX (NADPH oxidase) 1 & 4 inhibitors;
Farnesoid X receptor (FXR) agonists;
Fibroblast Growth Factor (FGF) 19 recombinant;
Fractalkine ligand inhibitors;
ileal sodium bile acid cotransporter inhibitors;
Monoclonal antibodies;
PPAR alpha agonists;
PPAR gamma agonists;
PPAR delta agonists;
PPAR alpha/gamma agonists;
PPAR alpha/delta agonists;
PPAR gamma/delta agonists; and
PPAR alpha/gamma/delta agonists or PPARpan agonists.

Illustrative apical sodium-codependent bile acid transporter inhibitors include, without limitation, odevixibat; volixibat; maralixibat formerly SHP-625; linerixibat; elobixibat and CJ-14199.

Illustrative bile acids include, without limitation, obeticholic acid and ursodiol (UDCA).

Illustrative cathepsin inhibitors include, without limitation, VBY-376; VBY-825; VBY-036; VBY-129; VBY-285; Org-219517; LY3000328; RG-7236 and BF/PC-18.

Illustrative CCR antagonists include, without limitation, cenicriviroc (CCR2/5 antagonist); PG-092; RAP-310; INCB-10820; RAP-103; PF-04634817 and CCX-872.

Illustrative CD40 inhibitors include, without limitation, FFp-104; xl-050; DOM-0800; XmAb-5485; KGYY-15; FFP-106; TDI-0028 and ABI-793.

Illustrative CD80 inhibitors include, without limitation, RhuDex; FPT-155; ToleriMab; galiximab; SCH-212394; IGM-001; ASP-2408 and SCH-204698.

Illustrative dual NOX (NADPH oxidase) 1 & 4 inhibitors include, without limitation, GKT-831 (formerly GKT137831) and GKT-901.

Illustrative Farnesoid X receptor (FXR) agonists include, without limitation, obeticholic acid; GS-9674; LJN-452; EDP-305; AKN-083; INT-767; GNF-5120; LY2562175; INV-33; NTX-023-1; EP-024297; Px-103 and SR-45023.

Illustrative Fibroblast Growth Factor 19 (FGF-19) recombinants include, without limitation, NGM-282.

Illustrative Fractalkine ligand inhibitors include, without limitation, E-601 1 and KAN-0440567. Illustrative ileal sodium bile acid cotransporter inhibitors include, without limitation, odevixibat; GSK-2330672; volixibat; CJ-14199 and elobixibat.

Illustrative monoclonal antibodies include, without limitation, bertilimumab; NGM-313; IL-20 targeting mAbs; fresolimumab (antiTGF3) formerly GC1008; timolumab formerly BTT-1023; namacizumab; omalizumab; ranibizumab; bevacizumab; lebrikizumab; epratuzumab; felvizumab; matuzumab; monalizumab; reslizumab and inebilizumab.

Illustrative PPAR alpha agonists include, without limitation, fenofibrate, ciprofibrate, pemafibrate, gemfibrozil, clofibrate, binifibrate, clinofibrate, clofibric acid, nicofibrate, pirifibrate, plafibride, ronifibrate, theofibrate, tocofibrate and SR10171.

Illustrative PPAR gamma agonists include, without limitation, Pioglitazone, deuterated pioglitazone, Rosiglitazone, efatutazone, ATx08-001, OMS-405, CHS-131, THR-0921, SER-150-DN, KDT-501, GED-0507-34-Levo, CLC-3001 and ALL-4.

Illustrative PPAR delta agonists include, without limitation, GW501516 (Endurabol or ({4-[({4-methyl-2-[4-(trifluoromethyl)phenyl]-1,3-thiazol-5-yl}methyl)sulfanyl]-2-methylphenoxy}acetic acid)) or MBX8025 (Seladelpar or {2-methyl-4-[5-methyl-2-(4-trifluoromethyl-phenyl)-2H-[1,2,3]triazol-4-ylmethylsylfanyl]-phenoxy}-acetic acid) or GW0742 ([4-[[[2-[3-fluoro-4-(trifluoromethyl)phenyl]-4-methyl-5-thiazolyl]methyl]thio]-2-methyl phenoxy]acetic acid) or L165041 or HPP-593 or NCP-1046.

Illustrative PPAR alpha/gamma agonists (also named glitazars) include, without limitation, saroglitazar, aleglitazar, muraglitazar, tesaglitazar and DSP-8658.

In addition to elafibranor, illustrative PPAR alpha/delta agonists include, without limitation, T913659.

Illustrative PPAR gamma/delta agonists include, without limitation, linoleic acid (CLA) and T3D-959.

Illustrative PPAR alpha/gamma/delta agonists (or “PPARpan agonists”) include, without limitation, IVA337, TTA (tetradecylthioacetic acid), bavachinin, GW4148, GW9135, bezafibrate, lobeglitazone, and CS038.

In a further embodiment, the present invention provides methods of treating a subject having PBC with a baseline plasma alkaline phosphatase (ALP) level of more than 1×ULN (Upper Limit Normal) and at most 1.67×ULN, in particular strictly less than 1.67×ULN, before receiving elafibranor, comprising the administration of elafibranor, in particular in the form of a pharmaceutical composition containing this compound, preferably an oral pharmaceutical composition containing this compound.

In a further embodiment, the present invention provides methods of treating a subject having PBC with a baseline plasma alkaline phosphatase (ALP) level of more than 1×ULN (Upper Limit Normal) and at most 1.67×ULN, in particular strictly less than 1.67×ULN, before receiving elafibranor, comprising the administration of elafibranor, in particular in the form of a pharmaceutical composition containing this compound, preferably an oral pharmaceutical composition containing this compound.

In another embodiment, the present invention also provides a kit for treating a subject having PBC with a baseline plasma alkaline phosphatase (ALP) level of more than 1×ULN (Upper Limit Normal) and at most 1.67×ULN, in particular strictly less than 1.67×ULN, before receiving elafibranor, comprising elafibranor, optionally in combination to another anti-cholestatic agent as described above.

In another embodiment, the present invention also provides a kit for treating a subject having PBC with a baseline plasma alkaline phosphatase (ALP) level of more than 1×ULN (Upper Limit Normal) and at most 1.67×ULN, in particular strictly less than 1.67×ULN, before receiving elafibranor, comprising elafibranor, optionally in combination to another anti-cholestatic agent as described above.

As previously mentioned, the present invention also provides the use of elafibranor in the preparation of a pharmaceutical composition, preferably an oral pharmaceutical composition, for treating PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

In a further embodiment, the present invention also discloses the use of elafibranor in the manufacture of a medicament for treating PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

In a further embodiment, the present invention also discloses the use of elafibranor in the manufacture of a medicament for preventing liver decompensation in a subject having PBC with a baseline plasma alkaline phosphatase (ALP) level of more than 1×ULN (Upper Limit Normal) and at most 1.67×ULN, in particular strictly less than 1.67×ULN, before receiving elafibranor.

The present invention further relates to a method for treating PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor, comprising administering a therapeutic effective amount of elafibranor.

In another embodiment of the invention, the invention also discloses a kit for treating PBC in a subject having PBC with a baseline plasma alkaline phosphatase (ALP) level of more than 1×ULN (Upper Limit Normal) and at most 1.67×ULN, in particular strictly less than 1.67×ULN, before receiving elafibranor, the kit comprising elafibranor.

The invention is further described with reference to the following, non-limiting, examples. The present invention will be better understood by referring to the following examples which are provided for illustrative purpose only and should not be interpreted as limiting in any manner the instant invention.

### EXAMPLES

### Example 1: Clinical Trial for PBC

A phase IIIb randomised, parallel-group, double-blind, placebo-controlled, two-arm study is conducted in patients with PBC and inadequate response or intolerance to ursodeoxycholic acid to evaluate the efficacy of 80 mg once daily elafibranor compared to placebo on normalisation of ALP in participants with PBC and an inadequate response or intolerance to UDCA with baseline ALP levels >1×ULN and <1.67×ULN.

### Primary Objectives

The primary objective is to evaluate the efficacy of daily oral administration of elafibranor 80 mg compared to placebo in adult participants with PBC on normalisation of ALP.

### Secondary Objectives

The secondary objectives are:
To evaluate the efficacy of daily oral administration of elafibranor 80 mg compared to placebo in adult participants with PBC on biochemical markers of response.
To evaluate the efficacy of daily oral administration of elafibranor 80 mg compared to placebo in adult participants with PBC on patient-reported outcomes.
To evaluate the safety and tolerability of daily oral administration of elafibranor 80 mg compared to placebo in adult participants with PBC.

### Exploratory

The exploratory objectives are:
To evaluate the effect of elafibranor 80 mg daily as compared to placebo on biomarkers: Liver tests, Non-invasive markers of fibrosis, Biomarkers of pruritus, Lipid parameters, PBC prognosis scores, biomarkers of inflammation and immune response, Biomarkers of bile acid synthesis, Bone mineral density.
To evaluate the efficacy of daily oral administration of elafibranor 80 mg compared to placebo in adult participants with PBC on clinical outcome events of interest.
To evaluate the PK of elafibranor and its metabolite GFT1007 in the participant population using a population PK approach.
To assess the relationship between PK and PD (efficacy and safety if applicable) endpoints.
To evaluate the association of biomarkers with clinical data on selected efficacy and safety endpoints, if applicable.

### Inclusion Criteria

1. Male or female participants age 218 years of age at the time of signing the informed consent.
2. Participants with a definite or probable diagnosis of PBC as demonstrated by the presence of ≥2 of the following three historical diagnostic criteria:
i. History of elevated ALP levels for 26 months prior to SV1.
ii. Positive AMA titres (≤1/40 on immunofluorescence or M2 positive by enzyme-linked immunosorbent assay) or positive PBC-specific antinuclear antibodies.
iii. Liver biopsy consistent with PBC.
3. ALP>1×ULN and <1.67×ULN based on two values obtained during screening. The interval between the two measurements should be at least 2 weeks.
4. Participants taking UDCA should have been on this medication for at least 6 months and at a stable dose for ≥3 months prior to SV1. Participants not taking UDCA or who are intolerant to UDCA may participate and should have taken the last dose of UDCA≥3 months prior to SV1.
5. Participants taking medications for management of pruritus (e.g. cholestyramine, rifampicin, naltrexone, sertraline or colchicine) must be on a stable dose for ≥3 months prior to screening.
6. Contraceptive use by men or women should be consistent with local regulations regarding the methods of contraception for those participating in clinical studies:
(a) Male participants must agree that, if their partner is at risk of becoming pregnant, they will use an effective method of contraception. The participant must agree to use contraception during the whole period of the study and for 30 days after the last dose of study intervention.
(b) Female participants are eligible to participate if she is not pregnant or breastfeeding, and one of the following conditions applies:
Is a woman of nonchildbearing potential (WONCBP)
OR
Is a woman of childbearing potential (WOCBP) and using a contraceptive method that is highly effective (with a failure rate of <1% per year), preferably with low user dependency, during the study intervention period and for at least 30 days after the last dose of study intervention. The investigator should evaluate the potential for contraceptive method failure (e.g. noncompliance, recently initiated) in relationship to the first dose of study intervention.

A WOCBP must have a negative highly sensitive pregnancy test (urine or serum, as required by local regulations) within 24 hours before the first dose of study intervention.
If a urine test cannot be confirmed as negative (e.g. an ambiguous result), a serum pregnancy test is required. In such cases, the participant must be excluded from participation if the serum pregnancy result is positive.
7. Capable of giving signed informed consent which includes compliance with the requirements and restrictions listed in the informed consent form (ICF) and in this protocol.

### Exclusion Criteria:

1. History or presence of other concomitant liver diseases including:
i. Primary sclerosing cholangitis.
ii. Autoimmune hepatitis (AIH) by simplified Diagnostic Criteria of the International Autoimmune Hepatitis Group ≥6, or if treated for an overlap of PBC with AIH, or if there is clinical suspicion and evidence of overlap AIH features, that cannot be explained alone by insufficient response to UDCA.
iii. Positive hepatitis B surface antigen (HBsAg). Participants with negative HBsAg and positive hepatitis B core antibody (HBcAb) may be eligible if hepatitis B virus deoxyribonucleic acid (HBV DNA) is negative.
iv. Hepatitis C virus (HCV) infection defined by positive anti-HCV antibody and positive HCV ribonucleic acid (RNA) (Note: Participants with positive anti-HCV antibody with documented previously treated HCV infection may be enrolled if a confirmatory HCV RNA is undetectable and sustained viral response has been documented).
v. Alcohol-associated liver disease
vi. Nonalcoholic steatohepatitis [NASH, also known as MASH (metabolic dysfunction-associated steatohepatitis]
2. Participants with known cirrhosis who have a Child-Pugh B or C score. Participants with cirrhosis with Child-Pugh A score are allowed.
3. History of liver transplantation.
4. History or presence of clinically significant hepatic decompensation, including:
i. Current placement on a liver transplant list, current model for end-stage liver disease (MELD)-Na score ≥12 due to hepatic impairment (Model for end stage liver disease including serum sodium (MELD-Na) will be calculated only when MELD >11).
ii. Evidence of complications of cirrhosis, including presence of ascites requiring treatment; history of variceal bleeding or related interventions (e.g. history of variceal band ligation or sclerotherapy, or transjugular intrahepatic portosystemic shunt (TIPS) placement); presence of hepatic encephalopathy Grade 2 or higher per West-Haven criteria; history or presence of spontaneous bacterial peritonitis. Note: Participants with Grade I oesophageal varices may be eligible to enrol.
iii. Hepatorenal syndrome (type 1 or II).
5. Known history of human immunodeficiency virus (HIV) infection.
6. Medical conditions that may cause non-hepatic increases in ALP (e.g. Paget's disease).
7. Evidence of any other unstable or untreated clinically significant immunological, endocrine, haematologic, gastrointestinal, neurological, or psychiatric disease as evaluated by the investigator; other clinically significant conditions that are not well controlled.
8. Cancer or non-hepatic medical condition with a life expectancy <2 years.
9. Known malignancy or history of malignancy within the last 2 years, except for successfully treated localised basal cell carcinoma or squamous cell carcinoma of the skin; or in-situ carcinoma of the uterine cervix.
10. History of hepatocellular carcinoma.
11. Alpha-foetoprotein (AFP)>20 ng/mL with 4-phase liver computed tomography (CT) or magnetic resonance imaging (MRI) scans suggesting presence of liver cancer.
12. Administration of the following medications is prohibited during the study, and prior to the study as per the timelines specified below:
i. Systemic (oral or parenteral) use within 3 months prior to SV1 of: fibrates, seladelpar, glitazones, obeticholic acid, azathioprine, cyclosporine, methotrexate, mycophenolate, or long-term systemic corticosteroids (parenteral and oral chronic administration only); potentially hepatotoxic drugs (including α-methyl-dopa, valproic acid, isoniazid or nitrofurantoin)
13. Participants with previous exposure to elafibranor.
14. Participants who are currently participating in, plan to participate in, or have participated in an investigational drug study or medical device study containing active substance within 30 days or 5 half-lives, whichever is longer, prior to SV1. If the previous study was for an experimental therapy being studied for potential benefit in PBC, and the potential therapeutic agent was proven to have no beneficial effect in PBC and there are no safety concerns, the participant may enrol after 30 days or 5 half-lives, whichever is longer. For therapeutic agents being studied for potential benefit in PBC for which it is still unclear if there may be a potential benefit, participants may enrol after 6 months.
15. Total bilirubin (TB)>2×ULN. Participants with Gilbert's syndrome are eligible with a TB above 2×ULN if direct bilirubin is <30% of TB.
16. Screening ALT and/or AST>5×ULN at SV1, or variability >40% based on two consecutive measurements. The interval between the two measurements should be at least 2 weeks.
17. CPK>2×ULN.
18. Platelet count <75,000/μL.
19. International normalised ratio >1.3 in the absence of anticoagulant therapy.
20. Estimated glomerular filtration rate (eGFR)<45 mL/min/1.73 m² per the Modification of Diet in Renal Disease (MDRD)-6 Study formula at SV1.
Note: In cases of decreased eGFR where the investigator believes the value may not be representative of the actual potential participant's eGFR, re-test after adequate hydration is allowed.
21. Significant renal disease, including nephritic syndrome, chronic kidney disease (defined as participants with evidence of significantly impaired kidney function or underlying kidney injury).

### Other Exclusions:

22. For female participants: known pregnancy, or has a positive serum pregnancy test, or is breastfeeding.
23. Regular alcohol intake in excess of the recommended limit of 2 standard drinks per day for men or 1 standard drink per day for women, where one standard measure corresponds to 10 g of alcohol.
24. History of alcohol abuse, or other substance abuse within 1 year prior to SV1.
25. Known hypersensitivity to the investigational product or to any of the excipients of elafibranor.
26. Mental instability or incompetence, such that the validity of informed consent or ability to be compliant with the study is uncertain.
27. Any other condition that, in the opinion of the investigator, would interfere with study participation or completion, or would put the participant at risk, including a potential participant assessed as being at high risk of noncompliance with the study.

### Randomisation

Patients who satisfy all eligibility criteria will be randomized in a 2:1 ratio to one of the following groups:
Elafibranor 80 mg/day; or
Placebo.

The randomisation ratio is stratified according to ALP at study entry (ALP>1×ULN to ≤1.33×ULN versus ALP>1.33×ULN to <1.67×ULN). When applicable, participants will continue to receive stable maintenance doses of UDCA throughout study participation.

The study consists of three periods:
An up to 8-week screening period;
A 52-week treatment period;
A 4-week follow-up period after the last dose of study intervention.

### Primary Endpoint

The primary endpoint is:
Percentage of participants with normalization of ALP (i.e. ALP≤1.0×ULN) at Week 52.

### Secondary Endpoint

The secondary endpoints are:
to assess at Week 4, Week 12, Week 24 and Week 36:
   Percentage of participants with normalization of ALP.
to assess at Week 4, Week 12, Week 24, Week 36 and Week 52:
   Absolute and percentage changes from baseline in ALP;
   Number and percentage of participants with normalization of ALP with reduction of ≥15% from baseline;
   Number and percentage of participants with ALP reduction of ≤40% from baseline;
   Number and percentage of participants with ALP<0.5×ULN;
   Absolute and percentage changes from baseline in TB;
   Number and percentage of participants with TB<0.7×ULN;
   Number and percentage of participants with normalization of ALP and TB<0.7×ULN;
   Number and percentage of participants with normalization of TB and ALP, defined as TB≤ULN and ALP≤ULN;
   Number and percentage of participants with complete biochemical response, defined as normal levels of TB, ALP, aminotransferases, albumin, and INR.
to assess through Week 52:
   Number and percentage of participants with moderate to severe pruritus at baseline (i.e. score ≥4) with a clinically meaningful response in PBC Worst Itch NRS, defined as ≥1.8-point reduction from baseline.
to assess at Week 4, Week 24, and Week 52:
   Change from baseline in 5-D itch score;
   Change from baseline in PGI-S scores;
   PGI-C scores;
   Change from baseline in PBC-40 QoL scores;
   Change from baseline in PROMIS Fatigue Short Form 7a scores.
For the duration of participation in the study:
   Number and percentage of participants who experience TEAEs, treatment related TEAEs, SAEs, and AESIs;
   Number and percentage of participants who develop PCSA and/or clinically significant changes from baseline in physical examination findings, vital signs, and ECG;
   Number and percentage of participants who develop PCSAs, and shifts from baseline in hematology, coagulation, chemistry, liver tests, renal tests (including urinalysis), and other laboratory tests and procedures.

### Exploratory Endpoints

to assess at Week 4, Week 12, Week 24, Week 36 and Week 52:
   Change from baseline in liver tests as measured by AST, ALT, GGT, conjugated bilirubin, albumin and INR.
to assess at Week 52:
   Change from baseline in LSM assessed by VCTE using Fibroscan®
   Change from baseline in non-invasive measures of hepatic fibrosis (such as by ELF test).
to assess at Week 12, Week 24, Week 36 and Week 52:
   Change from baseline in circulating markers of pruritus (such as IL-31, LPA and autotaxin).
to assess at Week 4, Week 12, Week 24, Week 36 and Week 52:
   Change from baseline in lipid parameters as measured by TC, LDL-C, HDL-C, calculated VLDL-C and TG.
To assess at Week 52:
   Change from baseline in PBC risk scores: UK PBC score and GLOBE score.
to assess at Week 24 and Week 52:
   Change from baseline in IgG and IgM.
   Change from baseline in circulating markers of inflammation (such as high-sensitivity C reactive protein, fibrinogen, haptoglobin, and tumour necrosis factor alpha).
to assess at Week 24 and Week 52:
   Change from baseline in C4 and FGF-19
to assess at Week 52:
   Change from baseline in bone mineral density.
   Number and percentage of participants with occurrence of any of the following clinical events of interest (and timing):
      All-cause mortality;
      Liver-related mortality;
      Liver transplantation;
      MELD-Na score 215 in participants with baseline MELD or MELD-Na score <12.
      Liver decompensation.
   Plasma concentrations and individual PK parameters after single administration and at steady state.
   Population PK parameters.
   (PK/PD) population and individual model parameters will be estimated if applicable.
   Biomarkers and clinical data analysis by modelling.

It is expected that elafibranor is better than placebo in preventing clinical outcome events showing disease worsening (including progression of disease leading to liver transplant or death), in participants with PBC and with a baseline plasma alkaline phosphatase (ALP) level of more than 1×ULN and at most 1.67×ULN, in particular strictly less than 1.67×ULN.

### Enumerated Embodiments

E1. Elafibranor for use for the treatment of Primary Biliary Cholangitis (PBC), wherein the subject to be treated is a subject having PBC with a baseline plasma alkaline phosphatase (ALP) level of more than 1×ULN (Upper Limit Normal) and strictly less than 1.67×ULN, before receiving elafibranor.
E2. Elafibranor for use for normalizing plasma ALP level in a subject having PBC, wherein the subject to be treated is a subject having PBC with a baseline plasma alkaline phosphatase (ALP) level of more than 1×ULN (Upper Limit Normal) and strictly less than 1.67×ULN, before receiving elafibranor.
E3. Elafibranor for use according to any one of embodiments E1 or E2, wherein the subject to be treated is identified by testing the ALP level of a potential subject and then selecting the subject having baseline plasma ALP level of more than 1×ULN (Upper Limit Normal) and strictly less than 1.67×ULN, before receiving elafibranor.
E4. Elafibranor for use according to any one of embodiments E1 to E3, wherein the subject responds at least partly to UDCA.
E5. Elafibranor for use according to any one of embodiments E1 to E4, wherein the subject does not respond adequately to UDCA.
E6. Elafibranor for use according to any one of embodiments E1 to E5, wherein the subject is intolerant to UDCA.
E7. Elafibranor for use according to any one of embodiments E1 to E6, for administration at a dose varying from 70 mg to 130 mg, preferably at a dose of 80 mg.
E8. Elafibranor for use according to any one of embodiments E1 to E7, in the form of a pharmaceutical composition, preferably an oral pharmaceutical composition.
E9. Elafibranor for use according to embodiments E8, wherein said composition is formulated in the form of injectable suspensions, gels, oils, pills, suppositories, powders, gel caps, capsules, aerosols or means of galenic forms or devices assuring a prolonged and/or slow release.
E10. Elafibranor for use according to any one of embodiments E1 to E9, for administration once a day.
E11. Elafibranor for use according to any one of embodiments E1 to E10, for oral administration once daily at a dose of 80 mg.
E12. Elafibranor for use according to any one of embodiments E1 to E11, in combination with another anti-cholestatic agent, preferably with UDCA.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed:

1. Elafibranor, or a pharmaceutical composition thereof, for use for the treatment of Primary Biliary Cholangitis (PBC), wherein the subject to be treated is a subject having PBC with a baseline plasma alkaline phosphatase (ALP) level of more than 1×ULN (Upper Limit Normal) and strictly less than 1.67×ULN, before receiving elafibranor.

2. Elafibranor for use according to claim 1, wherein the subject to be treated is identified by testing the ALP level of a potential subject and then selecting the subject having baseline plasma ALP level of more than 1×ULN (Upper Limit Normal) and strictly less than 1.67×ULN, before receiving elafibranor.

3. Elafibranor for use according to claim 1, wherein the subject responds at least partly to UDCA.

4. Elafibranor for use according to according to claim 1, wherein the subject does not respond adequately to UDCA.

5. Elafibranor for use according to claim 1, wherein the subject is intolerant to UDCA.

6. Elafibranor for use according to claim 1, for administration at a dose from 70 mg to 130 mg.

7. Elafibranor for use according to claim 1, wherein the elafibranor is in a pharmaceutical composition formulated for oral administration.

8. Elafibranor for use according to claim 7, for oral administration once daily at a dose of 80 mg.

9. Elafibranor for use according to claim 1, wherein said composition is formulated in the form of injectable suspensions, gels, oils, pills, suppositories, powders, gel caps, capsules, aerosols or means of galenic forms or devices assuring a prolonged and/or slow release.

10. Elafibranor for use according to claim 1, for administration once a day.

11. Elafibranor for use according to claim 1, in combination with another anti-cholestatic agent.

12. Elafibranor for use according to claim 11, wherein said another anti-cholestatic agent is UDCA.

13. Elafibranor, or a pharmaceutical composition thereof, for use for normalizing plasma ALP level in a subject having PBC, wherein the subject to be treated is a subject having PBC with a baseline plasma alkaline phosphatase (ALP) level of more than 1×ULN (Upper Limit Normal) and strictly less than 1.67×ULN, before receiving elafibranor.

14. Elafibranor for use according to claim 13, wherein the subject to be treated is identified by testing the ALP level of a potential subject and then selecting the subject having baseline plasma ALP level of more than 1×ULN (Upper Limit Normal) and strictly less than 1.67×ULN, before receiving elafibranor.

15. Elafibranor for use according to claim 13, for administration at a dose varying from 70 mg to 130 mg.

16. Elafibranor for use according to claim 13, wherein the elafibranor is part of a pharmaceutical composition formulated for oral administration.

17. Elafibranor for use according to claim 16, for oral administration once daily at a dose of 80 mg.

18. Elafibranor for use according to claim 13, wherein the elafibranor is part of a pharmaceutical composition formulated in the form of injectable suspensions, gels, oils, pills, suppositories, powders, gel caps, capsules, aerosols or means of galenic forms or devices assuring a prolonged and/or slow release.

19. Elafibranor for use according to claim 13, for administration once a day.

20. Elafibranor for use according to claim 13, wherein the subject responds at least partly to UDCA, does not respond adequately to UDCA, or is intolerant to UDCA.
